(19) United States
(12) Patent Application Publication
(10) Pub. No.: US 2024/0024650 A1
(43) Pub. Date: Jan. 25, 2024

(54) Venous Access Port Assembly With X-Ray Discernable Indicia

(71) Applicant: Medical Components, Inc., Harleysville, PA (US)

(72) Inventors: Raymond Bizup, Feasterville, PA (US); Kevin Sanford, Chalfont, PA (US); Christopher Linden, Vista, CA (US); Kenneth M. Zinn, Westport, CT (US); Timothy M. Schweikert, West Chester, PA (US)

(21) Appl. No.: 18/376,895

(22) Filed: Oct. 5, 2023

Related U.S. Application Data
(63) Continuation of application No. 17/122,251, filed on Dec. 15, 2020.
Application No. 17/122,251 is a continuation of application No. 15/251,122, filed on Aug. 30, 2016, now Pat. No. 10,874,842.
Application No. 15/251,122 is a continuation of application No. 13/088,762, filed on Apr. 18, 2011, now Pat. No. 9,517,329.
Application No. 13/088,762 is a continuation of application No. 12/175,182, filed on Jul. 17, 2008, now Pat. No. 8,021,324.
Application No. 12/175,182 is a continuation-in-part of application No. 11/725,287, filed on Mar. 19, 2007, now abandoned.
(60) Provisional application No. 60/961,133, filed on Jul. 19, 2007.
(60) Provisional application No. 60/852,591, filed on Oct. 18, 2006.

(51) Int. Cl. A61M 39/02; A61M 39/04

(52) U.S. Cl. CPC A61M 39/0208; A61M 39/04; A61M 2039/0045

(57) ABSTRACT

A venous access port assembly having a housing base with a discharge port, a septum, and a cap, with an interior reservoir. The housing base is provided with X-ray discernable indicia to identify an attribute of the assembly after its implantation and clearly appear on an X-ray of the patient in a manner informing the radiologist or technologist and the medical practitioner of that particular attribute. Such indicia can be depicted as cutouts through a disc of radiopaque material where the cutouts are in the form of alphabetical letters such as “CT”, or can be a set of discrete elements of radiopaque material, that are affixed along the bottom surface of the housing base or embedded within the thickness of the bottom housing wall.

### CROSS-REFERENCE TO RELATED APPLICATIONS

This application is a continuation of U.S. patent application Ser. No. 17/122,251, filed Dec. 15, 2020, which is a continuation of U.S. patent application Ser. No. 15/251,122, filed Aug. 30, 2016 (now U.S. Pat. No. 10,874,842, issued Dec. 29, 2020), which is a continuation of Ser. No. 13/088,762, filed Apr. 18, 2011 (now U.S. Pat. No. 9,517,329, issued Dec. 13, 2016), which is a continuation of U.S. patent application Ser. No. 12/175,182, filed Jul. 17, 2008 (now U.S. Pat. No. 8,021,324, issued Sep. 20, 2011), which claims the benefit of U.S. provisional application Ser. No. 60/961,133, filed Jul. 19, 2007, and a continuation-in-part of U.S. patent application Ser. No. 11/725,287, filed Mar. 19, 2007 (now abandoned), which claims the benefit of U.S. provisional application Ser. No. 60/852,591, filed Oct. 18, 2006, the contents of all said applications are incorporated herein by reference.

### FIELD OF THE INVENTION

This relates to the field of medical devices and more particularly to venous access ports for the infusion of fluids into a patient and/or withdrawal of fluids from a patient.

### BACKGROUND OF THE INVENTION

Venous access ports for the infusion and/or withdrawal of fluids from a patient are well-known, secured to the proximal end of an implanted catheter. These ports are typically used for drug infusion or for withdrawal of small amounts of blood, where large flows of fluid are not required. The ports are assemblies of a needle-impenetrable housing with a discharge port in fluid communication with a catheter and a reservoir within the port housing, and provide a subcutaneous self-sealing septum that defines an access site for multiple needle sticks through the covering skin tissue of the patient, through the septum, and into the reservoir, without the need to continuously search for new access sites. Examples of such ports are disclosed, for example, in U.S. Pat. Nos. 4,704,103; 4,762,517; 4,778,452; 5,185,003; 5,213,574 and 5,637,102.

It is desired to provide a venous access port assembly that provides for a radiologist, radiology technologist, nurse, and ultimately a medical practitioner to be able to discern an important property of the port assembly after the port assembly has been implanted into a patient.

### SUMMARY OF THE INVENTION

An embodiment of the present invention is related to a venous access port having a housing and a septum, providing an interior reservoir and a passageway extending from the reservoir through a stem of a discharge port to establish fluid communication with a proximal end of a catheter lumen to which the port assembly is secured prior to placement of the assembly into a patient. The port may optionally have more than one reservoir and associated septum. An embodiment of the present invention includes the incorporation of X-ray discernable indicia onto a venous access port that is discernible under X-ray examination to provide information concerning the nature or key attribute of the venous access port, so that the practitioner, subsequent to the date of implantation thereof, can determine that nature or key attribute under X-ray examination. One such key attribute in particular would be for example that the venous access port is rated to be used for power injection such as of contrast fluid, wherein for example the letters “CT” (for “computed tomography”, or “contrast enhanced computed tomography”) would be provided that are of radiopaque material, or are cutouts through radiopaque material. The attribute in this example is the property of the port being adapted to withstand high pressures that are used for injection of contrast fluid into a patient, and the letters “CT” would be understood in medical practice to indicate that the port is suitable for the high pressure injection of contrast fluid.

In one embodiment, a disc of radiopaque material includes cutouts of letters “CT” (although other indicia may be utilized) through the body of the disc. In another embodiment, discrete letters “CT” (although other indicia may be utilized) are provided of radiopaque material. With either embodiment, the disc or letters may be insert molded within the housing base bottom wall, or they may be affixed to the bottom surface of the housing base, preferably within complementary recesses thereinto, in such a manner that the letters “CT” are readable from above the port assembly in an X-ray.

### BRIEF DESCRIPTION OF THE DRAWINGS

The accompanying drawings, which are incorporated herein and constitute part of this specification, illustrate embodiments of the invention, and, together with the general description given above and the detailed description given below, serve to explain the features of the embodiments of the invention. In the drawings:

FIGS. 1 and 2 are an isometric view and a plan view of a venous access port, in accordance with an exemplary embodiment of the present invention;

FIGS. 3 and 4 are cross-sectional views of the port of FIGS. 1 and 2 taken along lines 3-3 and lines 4-4 of FIG. 1, respectively;

FIG. 5 is an isometric view of the needle-impenetrable housing base of the venous access port of FIG. 1;

FIGS. 6 and 7 are transverse cross-sectional and longitudinal cross-sectional views of the housing base of FIG. 5;

FIG. 8 is an isometric view of a first embodiment of X-ray discernable indicia, being a disc of radiopaque material having letters cut out thereof;

FIGS. 9 to 11 are bottom, cross-sectional, and top views of the port assembly of FIGS. 1 to 7 having the disc of FIG. 8 affixed to the housing base of FIGS. 6 and 7 and within a shallow recess into its bottom surface, with silicone covering molded thereover, and the indicia being shown in dashed lines in FIGS. 9 and 11;

FIG. 12 is a cross-sectional view of an alternate embodiment of the housing base having the disc of FIG. 8 insert molded embedded within the bottom wall of the base;

FIG. 13 is an isometric view of a second embodiment of radiopaque indicia, comprising a set of discrete letters of radiopaque material;

FIGS. 14 and 15 are a bottom view and a cross-sectional view of a port assembly of FIGS. 1 to 7 having the discrete letters of FIG. 13 insert molded into the bottom wall of the housing base, with FIG. 15 taken along lines 15-15 of FIG. 13; and

FIGS. 16 to 18 are a bottom view, cross-sectional view, and an isometric bottom view of a port assembly of FIGS. 1 to 7 having the discrete letters of FIG. 13 affixed to the bottom surface of the housing base, shown within respective recesses thereinto, with a silicone covering molded thereover, with FIG. 17 taken along lines 17-17 of FIG. 16.

### DETAILED DESCRIPTION OF THE INVENTION

Certain terminology is used herein for convenience only and is not to be taken as a limitation on the present invention. The terms “distal” and “proximal” refer, respectively, to directions closer to and away from the insertion tip of a catheter in an implantable catheter assembly. The terminology includes the words specifically mentioned, derivatives thereof, and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

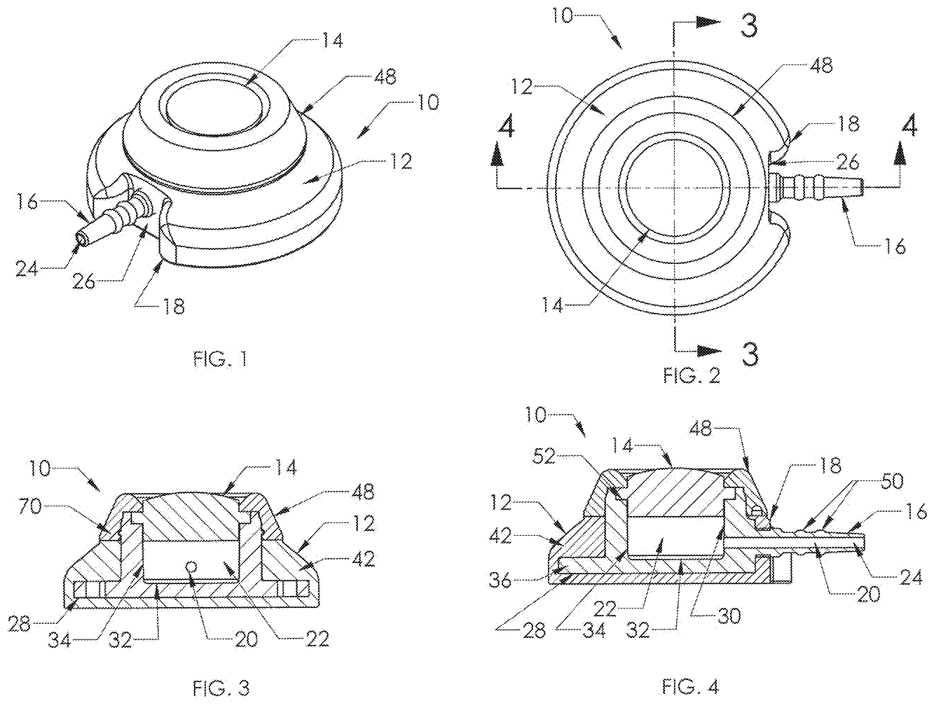

Venous access port assembly 10 of FIGS. 1 to 4 includes a housing 12 and a septum 14, with a discharge port 16 extending from a distal end 18 of the port assembly 10 to be attached securely and sealingly to the proximal end of a catheter (not shown). A passageway 20 extends from the interior reservoir 22 to the distal tip opening 24 of discharge port 16. A recess 26 is seen to be provided along both sides of discharge port 16, facilitating insertion of the discharge port 16 into the catheter lumen and providing a clearance for a locking sleeve or clamp (not shown) utilized to compress the catheter lumen wall against the exterior surface of the discharge port 16 for assured sealed connection of the catheter with the port assembly 10.

With reference now to FIGS. 3 to 7, the interior of the port assembly 10 is shown to provide an interior reservoir 22. Housing 12 is shown to include a housing base 28 of needle-impenetrable material that includes a well 30 having a bottom floor 32 and side walls 34 that define the interior reservoir 22 beneath septum 14. Bottom floor 32 may be convex or elevated (not shown) toward the center of the reservoir, if desired. Housing base 28 includes a base flange 36 extending radially outwardly from the bottom of well 30, and base flange 36 includes openings 38,40 that serve to enable suturing to the patient upon placement of the venous access port and the attached catheter into the patient.

As shown in FIGS. 3 and 4, a skirt 42 is overmolded about housing base 28 and may be of silicone elastomer. It is seen that skirt 42 encapsulates the outer surfaces of the bottom wall 44 and the bottom portion of the side walls 46 of housing base 28, and is shown to fill in the suture holes 38,40; but since the material is silicone elastomer, suturing is possible since the suturing needle can easily be inserted through the material of skirt 42 and through the suture holes, and thereafter the filled openings provide minimal opportunity for ingrowth of patient tissue into the openings.

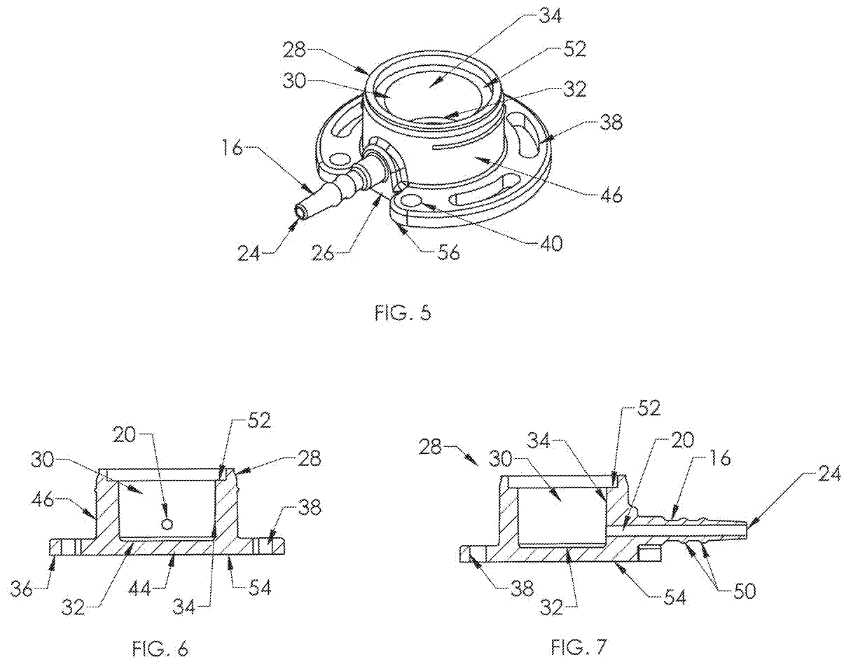

Also seen in FIGS. 1 to 4 is cap 48, which secures to housing base 28 to in turn secure septum 14 in position in the port assembly 10. Preferably, skirt 42 is insert molded onto base flange 36 of housing base 28 before cap 48 is secured to the upper portion of housing base 28 to secure the septum in position. It is seen in FIGS. 4 and 7 that discharge port 16 is integral with housing base 28 as is preferable. Discharge port 16 is shown to have a pair of annular ridges 50 that facilitate with the mechanical connection of the catheter proximal end with the port assembly 10. Housing base 28 includes a septum seat 52 extending into the top of well 30, into which a flange of the septum will be seated, preferably under radially inward compression. Housing base 28 has a bottom outer surface 54.

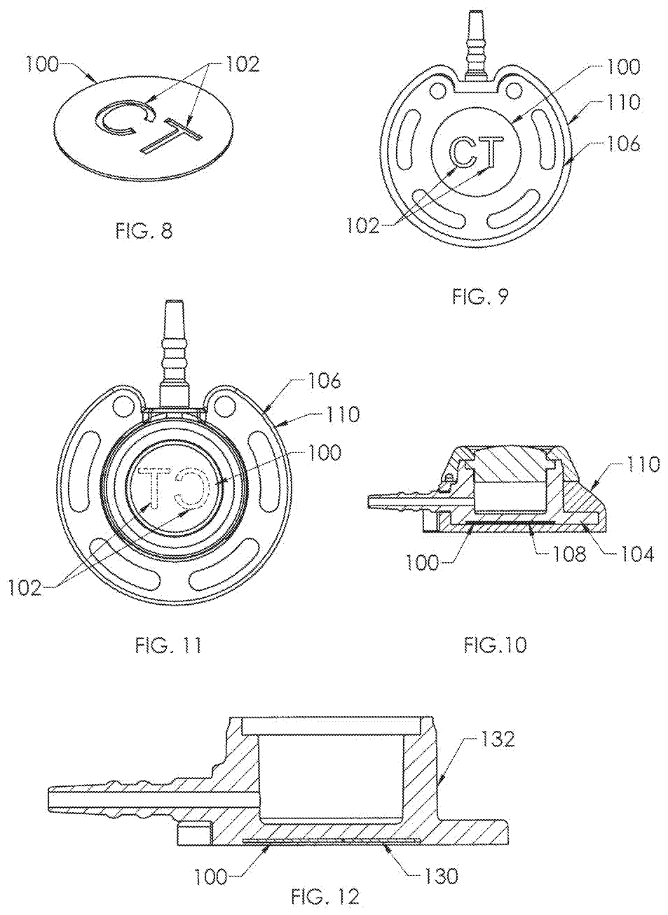

FIG. 8 shows a first embodiment of a component of radiopaque material of the present invention in the form of a disc 100, such as of titanium. Cutouts 102 are formed through the disc body, shown in FIG. 8 as the alphabetical letters “CT”. Disc 100 is affixed to the bottom surface 104 of housing base 106 in FIGS. 9 and 10, preferably within a complementary shallow recess 108 thereinto. A skirt 110 of silicone material is molded over the housing base, and is transparent so that the letters “CT” are visible from below but in a mirror-image orientation on the bottom outer surface of the housing base (FIG. 9) so that the indicia would appear as “CT” when the X-ray is viewed (FIG. 11), easily discerned by the radiologist or technologist. Centering of the indicia within the region directly beneath the reservoir and septum minimizes any obscuring by the structure of the venous access port assembly, and the indicia may also be easily discernable should the port assembly be at an angle from the horizontal plane of the X-ray.

In FIG. 12, an alternate embodiment of the present invention is shown, in which the disc 100 of FIG. 8 is embedded within the thickness of the bottom wall 130 of the housing base 132, and the X-ray would appear very similar to that shown in FIG. 11 but the indicia would not be visible from below the housing base or the port assembly.

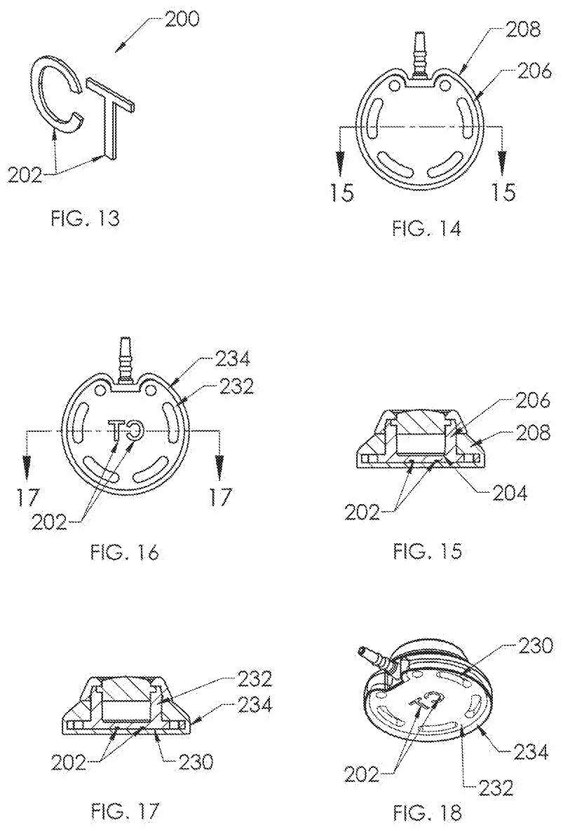

A second embodiment of X-ray discernable indicia 200 is shown in FIG. 13, and is utilized in the port assemblies of FIGS. 14 to 18. In FIG. 13, the indicia comprise a set of discrete indicia elements 202 of radiopaque material, such as being stamped from a sheet of titanium. Again, as is preferred, the indicia comprise the alphabetical letters “C” and “T” and are utilized together as a set. In FIGS. 14 and 15, the discrete elements are embedded into the thickness of the bottom wall 204 of housing base 206, so that they would not be visible from below (see FIG. 14) even though the silicone overmolded skirt 208 is transparent. However, the discrete letters 202 would clearly be visible on an X-ray very similarly to the port assembly shown in FIG. 11. Another manner of using discrete letters 202 is depicted in FIGS. 16 to 18, in which the letters 202 are insert molded along the bottom surface 230 of housing base 232 and recessed thereinto, preferably. With this variant, the radiopaque material may be titanium or may be, for example, silicone material having barium sulfate filler. In this case the mirror-image of “CT” would be visible from below as depicted in FIG. 18 after the silicone overmolding of skirt 234 about the exterior of housing base 232.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. An implantable venous access port assembly, comprising:
a needle-penetrable septum;
a housing securing the needle-penetrable septum; and
a radiopaque material affixed to the housing wherein the radiopaque material produces a contrasting image under X-ray examination,
wherein the radiopaque material comprises at least one cutout.

2. The port assembly of claim 1, wherein at least a portion of the radiopaque material is at least partially embedded within the housing.

3. The port assembly of claim 1, wherein at least a portion of the radiopaque material is indictive of an attribute of the access port assembly.

4. The port assembly of claim 1, wherein the radiopaque material further comprises a generally planar portion recessed into a bottom surface of a bottom wall of the housing, the generally planar portion including the at least one cutout.

5. The port assembly of claim 4, wherein the attribute is a pressure property of the port assembly.

6. The port assembly of claim 5, wherein the pressure property is the port assembly is being rated for power injection.

7. The port assembly of claim 5, wherein the pressure property is the port assembly can withstand pressures for injection of contrast fluid.

8. The port assembly of claim 1, wherein the radiopaque material comprises titanium.

9. The port assembly of claim 1, wherein the radiopaque material comprises a polymer with a radiopaque filler.

10. The port assembly of claim 1, wherein the housing further comprises a skirt formed from radiotransparent material molded about at least a portion of the housing.

11. The port assembly of claim 1, wherein the at least one cutout is formed in a shape of at least one alphabetical letter.

12. The port assembly of claim 11, wherein the at least one alphabetical letter comprises at least one of “C” and “T”.

13. The port assembly of claim 1, wherein at least a portion of the housing is made of polysulfone resin.

14. The port assembly of claim 1, wherein the at least a portion of the radiopaque material is centered within the region directly beneath the septum.

15. The port assembly of claim 1, wherein the housing and the septum define a reservoir, and at least a portion of the radiopaque material is projecting outward of a periphery of the reservoir.

16. The port assembly of claim 1, wherein the housing comprises a material of a different radiopacity from the radiopaque material.

17. The port assembly of claim 16, wherein the housing comprises a material of less radiopacity compared to the radiopaque material.

18. The port assembly of claim 1, wherein the housing comprises a material of a substantially the same radiopacity as the radiopaque material.
